(19) United States
(12) Patent Application Publication
(10) Pub. No.: US 2016/0089571 A1
(43) Pub. Date: Mar. 31, 2016

(54) REHABILITATION MONITORING DEVICE

(71) Applicant: TL TECHNOLOGIES LLC, Mableton, GA (US)

(72) Inventors: Tyler Wesley, Mableton, GA (US); Luke Edwards, Mableton, GA (US); Anish Joseph, Lilburn, GA (US)

(21) Appl. No.: 14/787,158

(22) PCT Filed: Apr. 23, 2014
(86) PCT No.: PCT/US14/35174

§ 371 Date: Oct. 26, 2015

Related U.S. Application Data
(60) Provisional application No. 61/815,425, filed on Apr. 24, 2013.

(51) Int. Cl. A63B 24/00; A61B 5/11; A61B 5/107

(52) U.S. Cl. CPC A63B 24/0062; A61B 5/1079; A61B 5/1071; A61B 5/1121

(57) ABSTRACT

A wearable rehabilitation monitoring device is disclosed that can be used by patients and medical professionals to monitor progress and compliance with at-home rehabilitation programs. Also disclosed is a method for monitoring rehabilitation by a subject that involves collecting data from a rehabilitation monitoring device worn by the subject during at-home rehabilitation exercise.

### CROSS-REFERENCE TO RELATED APPLICATIONS

This application claims benefit of U.S. Provisional Application No. 61/815,425, filed Apr. 24, 2013, which is hereby incorporated herein by reference in its entirety.

### TECHNICAL FIELD

This invention relates generally to wearable rehabilitation monitoring devices and systems and methods related to same.

### BACKGROUND

After accidents or strokes, persons often need a prolonged rehabilitation process in an attempt to recapture some or all of the body function damaged in the accident or stroke. Such rehabilitation may include physical rehabilitation in which damaged or unused muscles, nerves and/or joints are exercised by physical therapy. This typically involves the personal attention of a physical therapist that monitors and instructs a patient in the performance of certain exercises. As a result of rising health care costs, rehabilitation programs are often being performed in a patient's home without a visiting therapist being physically present. However, at-home rehabilitation programs suffer from compliance and monitoring deficiencies. The physical therapist has no way to determine whether the patient followed the rehabilitation program and must rely on the office visit to ascertain progress.

### SUMMARY

A wearable rehabilitation monitoring device is disclosed that can be used by patients and medical professionals to monitor progress and compliance with at-home rehabilitation programs. For example, the disclosed device can monitor rehabilitation exercises by a subject involving a synovial joint. Non-limiting examples of synovial joints include the knee, ankle, shoulder, elbow, hand, and wrist of the subject. In some embodiments, the disclosed device contains a goniometer configured to measure flexion/extension kinematics of the joint; a dynamometer configured to detect flexor strength, extensor strength, or a combination thereof, of the joint; a wearable attachment portion configured to operably position the goniometer and dynamometer relative to the joint; and an interface configured to receive data from the goniometer and dynamometer.

The disclosed rehabilitation monitoring device can be in any wearable form configurable for monitoring range of motion and strength about a joint. For example, the rehabilitation monitoring device can be in the form of a glove or a brace. Therefore, in some cases, the attachment portion comprises a glove for securing the device across a metacarpophalangeal joint in the hand of the subject. When used, a brace can be either rigid or flexible. For example, the brace can have a frame with hinges. Alternatively, the brace can be a flexible sheath. Suitable sensors can be selected based on the chosen structure.

The disclosed rehabilitation monitoring device can further comprise a sensory feedback portion affixed to the wearable attachment. In some embodiments, the sensory feedback portion is configured to generate a visible cue, audible cue, haptic cue, or combination thereof, to the subject.

The goniometer in the disclosed rehabilitation monitoring device can in some embodiments be any sensor suitable for measuring range of motion about the joint. Non-limiting examples include Hall effect, inductive, magnetoresistive, and potentiometric rotary position sensors. Further examples include force sensitive resistors. In some embodiments, the goniometer comprises a flexion sensor, such as a fibre-optic bend sensor.

The dynamometer in the disclosed rehabilitation monitoring device can in some embodiments be any sensor suitable for measuring strength about the joint. Non-limiting examples include pressure sensors and force sensitive resistors.

The interface in the disclosed rehabilitation monitoring device can in some embodiments be configured to transmit data from the goniometer and/or dynamometer to a computing device. For example, the computing device can be a personal computer or a mobile computing device (e.g., smartphone or tablet). The interface can therefore comprise a computer readable medium configured to store the data for later transmission.

In some implementations, the interface and/or computing device contains a processor and memory configured with computer-executable instructions that when executed by the processor cause the processor to analyze the subject's daily rehabilitation regimen. For example, the instructions can cause the processor to calculate compliance by the subject to a daily rehabilitation regimen. The instructions can cause the processor to generate a cue in the sensory feedback portion when the subject complies with a set range of motion, a set strength, or a combination thereof. The instructions can cause the processor to generate a cue in the sensory feedback portion when the subject complies with a set number of repetitions. The instructions can cause the processor to generate a cue in the sensory feedback portion after a set time has elapsed.

The disclosed rehabilitation monitoring device can further comprise a resistance portion that inhibits flexion, extension, or a combination thereof. In some cases, the resistance portion is located within the dynamometer. Non-limiting examples of resistance portions include springs, clamps, and locks. In some implementations, the resistance portion is isokinetic. For example, the resistance portion can have a motor to provide isokinetic resistance to the force applied and then use a computer to keep the motion smooth. Other ways of developing isokinetic motion include hydraulic systems (water or oil commonly), clutch based systems, friction based systems, and elastic resistance machines.

Also disclosed is a method for monitoring rehabilitation by a subject that involves collecting data from a rehabilitation monitoring device worn by the subject during at-home rehabilitation exercise, wherein the rehabilitation monitoring device comprises a goniometer positioned to measure flexion/extension kinematics of the joint and a dynamometer configured to detect flexor strength, extensor strength, or a combination thereof, of the joint; and analyzing the data to determine rehabilitation compliance, progress, or a combination thereof, by the subject. For example, the rehabilitation can involve stroke rehabilitation and/or joint rehabilitation.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a perspective view of a rehabilitation monitoring device in the form of a glove for placement on a subject's hand;

FIG. 2A is a perspective view of a rehabilitation monitoring device in the form of a brace for placement on a subject's knee;

FIG. 2B is a top view of a rehabilitation monitoring device in the form of a brace for placement on a subject's knee;

FIG. 2C is a side view of a rehabilitation monitoring device in the form of a brace for placement on a subject's knee;

FIG. 2D is a front view of a rehabilitation monitoring device in the form of a brace for placement on a subject's knee;

FIG. 3 is a block diagram illustrating a rehabilitation monitoring system according to implementations discussed herein; and

FIG. 4 is a block diagram of an example computing device.

### DETAILED DESCRIPTION

A wearable rehabilitation monitoring device is disclosed that can be used by patients and medical professionals to monitor progress and compliance with at-home rehabilitation programs. The device generally contains a wearable attachment portion configured to operably position the goniometer and dynamometer relative to the joint. The device can be in any wearable form configurable for monitoring range of motion and strength about a joint. For example, the rehabilitation monitoring device can be in the form of a glove or a brace. The device also generally contains a goniometer configured to measure flexion/extension kinematics of the joint; a dynamometer configured to detect flexor strength, extensor strength, or a combination thereof, of the joint; and an interface configured to receive data from the goniometer and dynamometer.

The disclosed rehabilitation monitoring device can further comprise a sensory feedback portion affixed to the wearable attachment. In some embodiments, the sensory feedback portion is configured to generate a visible cue, audible cue, haptic cue, or combination thereof, to the subject. Non-limiting examples include lights (e.g., LED lights), speakers, buzzers, or vibrators.

The disclosed rehabilitation monitoring device can further comprise a resistance portion that inhibits flexion, extension, or a combination thereof. In some cases, the resistance portion is located within the dynamometer. Non-limiting examples of resistance portions include springs, clamps, and locks. In some implementations, the resistance portion is isokinetic. For example, the resistance portion can have a motor to provide isokinetic resistance to the force applied and then use a computer to keep the motion smooth. Other ways of developing isokinetic motion include hydraulic systems (water or oil commonly), clutch based systems, friction based systems, and elastic resistance machines.

The interface can in some embodiments be configured to transmit data from the goniometer and/or dynamometer to a computing device. For example, the computing device can be a personal computer or a mobile computing device (e.g., smartphone or tablet). The interface can therefore comprise a computer readable medium configured to store the data for later transmission.

In some implementations, the interface and/or computing device contains a processor and memory configured with computer-executable instructions that when executed by the processor cause the processor to analyze the subject's daily rehabilitation regimen. For example, the instructions can cause the processor to calculate compliance by the subject to a daily rehabilitation regimen. In some cases, the instructions cause the processor to generate a cue in the sensory feedback portion when the subject complies with a set range of motion, a set strength, or a combination thereof. In some cases, the instructions cause the processor to generate a cue in the sensory feedback portion when the subject complies with a set number of repetitions. In some cases, the instructions cause the processor to generate a cue in the sensory feedback portion after a set time has elapsed. These and other instructions can be used together to provide feedback to the subject user and/or information to a medical professional relating to progress and compliance of the subject.

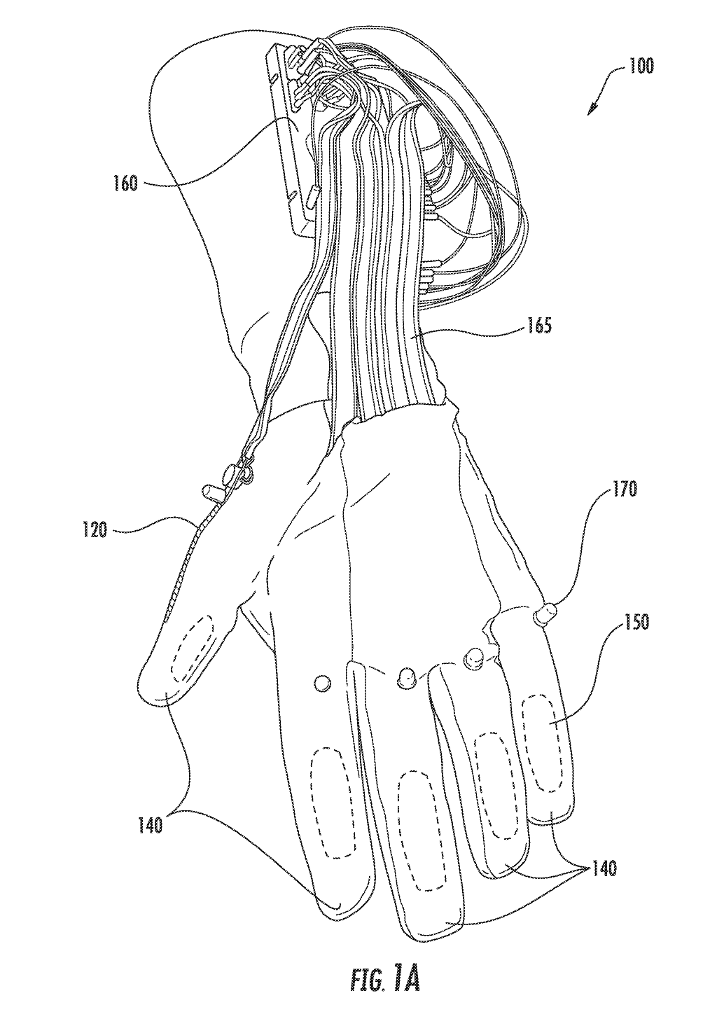

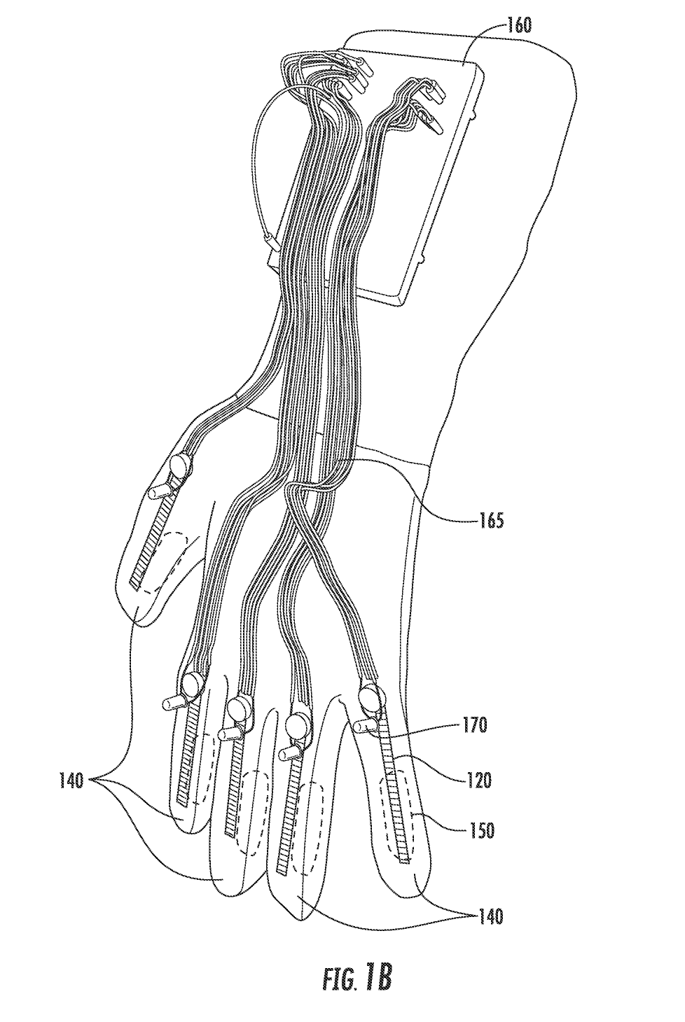

Referring now to FIGS. 1A and 1B, an embodiment of the disclosed rehabilitation monitoring device 10 in the form of a glove 100 for placement on a subject's hand is shown. The glove 100 can have separate sheaths 140 for each finger and thumb. The glove 100 can be sized to be universal, i.e., useable on either hand, or adjustable, and, optionally, has its fingertip portions removed, to accommodate different hand sizes.

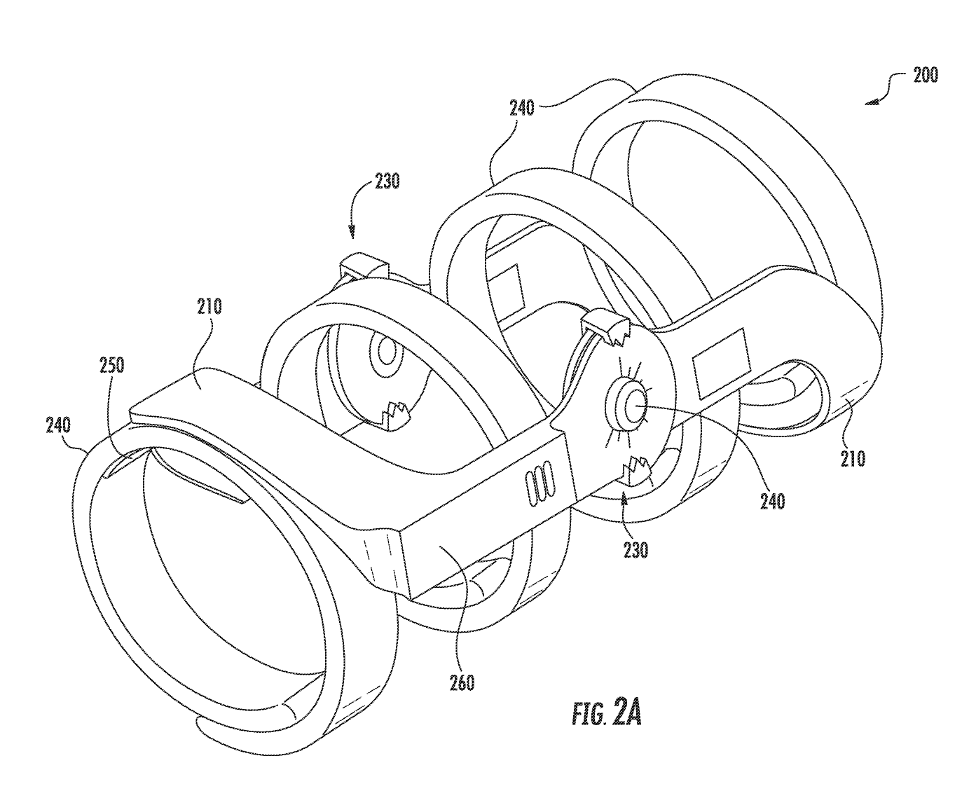

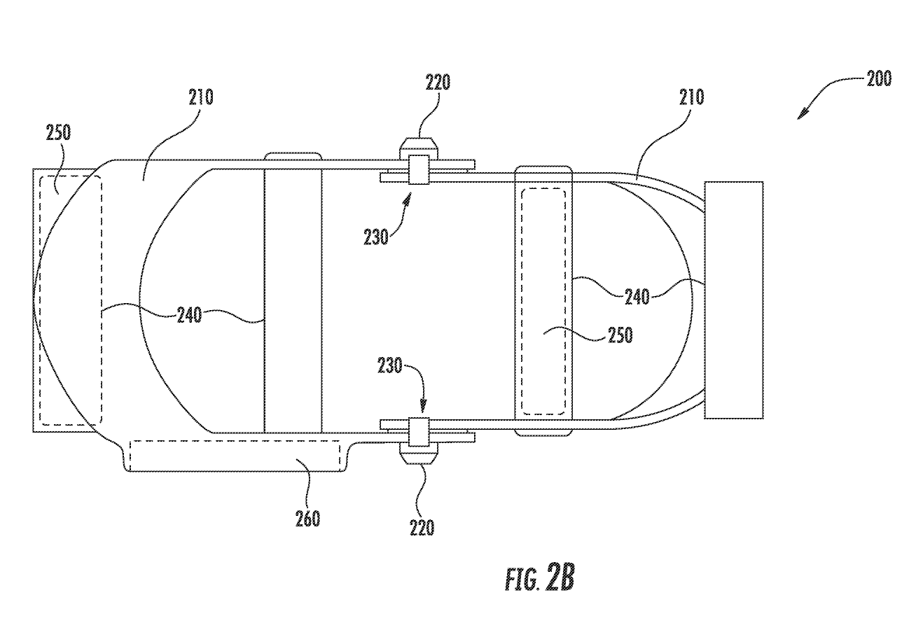

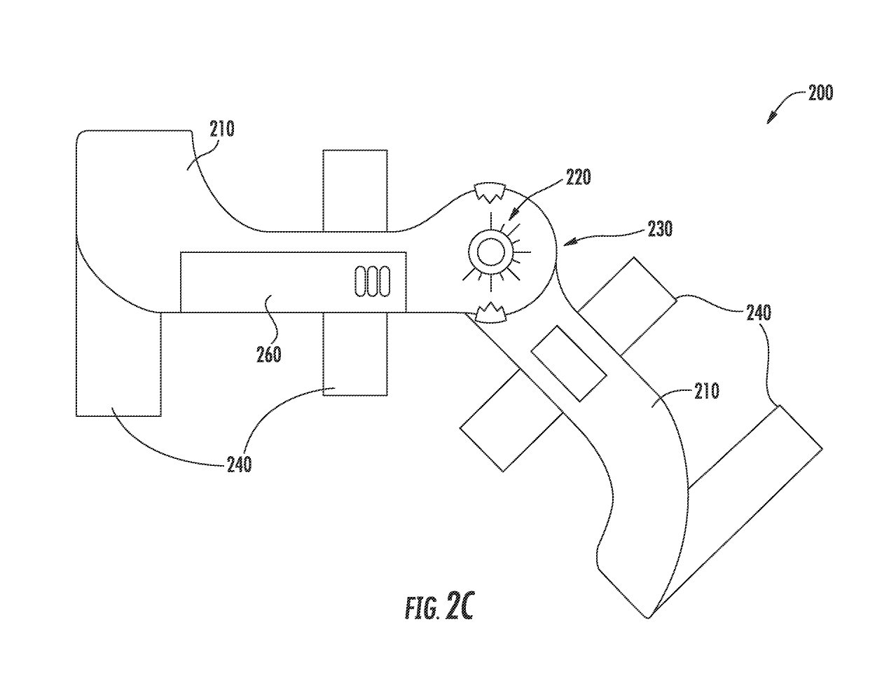

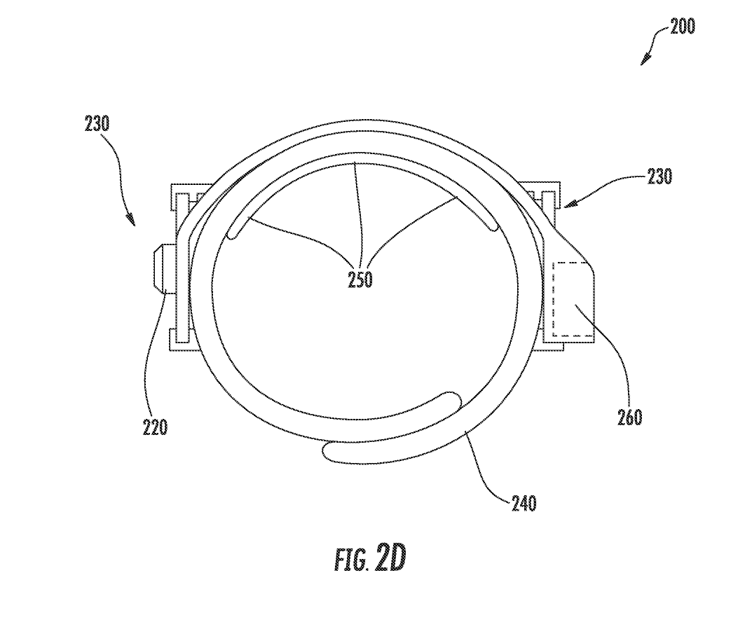

Referring to FIGS. 2A to 2D, an embodiment of the disclosed rehabilitation monitoring device 10 in the form of a brace 200 for placement on a subject's knee is shown. The brace 200 can include a frame 210 and straps 240 sized and positioned to secure the brace 200 to a subject's knee. However, in some cases, the brace 200 comprises a flexible sheath and lacks a rigid brace. When a frame is used, the brace 200 can further include a pair of hinges 230 operably positioned to align with the pivot axis of the subject's knee.

The rehabilitation monitoring device, e.g., glove 100 or brace 200, can include a goniometer for configured to measure flexion/extension kinematics of the joint. In FIGS. 1A and 1B, the goniometer is shown as a flex sensor 120 integrated along the finger and thumb sheaths 140 and operably positioned to detect flexion/extension kinematics of the fingers in the glove 100. In FIGS. 2A to 2D, the goniometer is shown as a potentiometer 220 integrated into the hinge 220 of a brace 200.

Goniometer sensing devices (“Goniometers”) are provided to sense movement, e.g., bending, flexion, and so forth. The bend or flexion of a human synovial joint can be measured using various methods.

In some embodiments, the goniometer involves a bend sensor. Electronic bend sensors use physical geometries and material properties to alter an electrical signal in proportion with angle or pressure. Bend radius and bend angle affect sensor output voltage. Other types of bend sensors include optical fiber sensors, electromechanical sensors, and mechanical measurement devices.

Optical bend sensors typically include a light source that is coupled to a light detector using, for example, an optical fiber. As the fiber bends, less light traverses the length of the fiber due to total internal reflection (TIR). For example, at higher bend angle, relatively few rays strike the detector and more rays exit the fiber at large angles. The optical method provides a repeatable measurement of a bend angle. Other optical technologies improve on the concept by using multiple fibers in a bundle or by pre-bending the fiber in a certain direction, and are therefore able to measure direction of bend as well as magnitude.

A class of angle measurement sensors exists that relies on mechanical means such as the tension of a cable disposed inside a rigid tube, or the relative position of members in an armature.

Hall effect sensors are switches that are activated in the presence of a magnetic field such as generated by a magnetic field-producing device, e.g., a magnet.

The sensor contains a capacitor that generates an electrical current and a magnetic field perpendicular thereto. The magnetic charges generated follow a straight line except when in proximity of a magnetic field at which time the path of the charge becomes non-linear, i.e., curves, and accumulates on one face of the sensor. The distance at which the magnetic field causes the sensor to act like a switch is a function of the strength of the magnetic field and, therefore, the magnet, and the current density specified by the sensor.

The rehabilitation monitoring device, e.g., glove 100 or brace 200, can include a dynamometer configured to detect flexor strength, extensor strength, or a combination thereof, about the joint. In FIGS. 1A and 1B, the dynamometer is shown as pressure pads 150 integrated into one or more of the sheaths 140. For example, the pressure pads 150 can be operably positioned to contact the palmar surface of the patient's limbs to measure the force applied during finger flexion. The pressure pads 150 can also be operably positioned to contact the dorsal surface of the patient's limbs to measure the force applied during finger extension. In FIGS. 2A to 2C, the dynamometer is also shown as pressure pads 250 integrated into one or more of the straps 240. For example, the pressure pads 250 can be operably positioned to contact the anterior surface of the patient's limbs to measure the force applied during limb extension. The pressure pads 250 can also be operably positioned to contact the posterior surface of the patient's limbs to measure the force applied during limb flexion. An alternative to pressure pads includes force sensitive resistors (FSRs).

The rehabilitation monitoring device, e.g., glove 100 or brace 200, can include a sensory feedback portion affixed to the wearable attachment. In some embodiments, the sensory feedback portion is configured to generate a visible cue, audible cue, haptic cue, or combination thereof, to the subject. Non-limiting examples include lights (e.g., LED lights), speakers, buzzers, or vibrators. In FIGS. 1A and 1B, the sensory feedback portion is shown as LED lights 170 integrated into the flex sensor 120.

The rehabilitation monitoring device, e.g., glove 100 or brace 200, can further include an interface 160, 260 configured to receive data from the goniometer and/or dynamometer. For example, in some embodiments, the interface 160, 260 is configured to transmit the data to a computer. For example, the interface can contain a wireless receiver (e.g., a Bluetooth®, infrared, IEEE 802.11 (Wi-Fi™), or IEEE 802.15). The interface 160, 260 can also contain an interface connector, such as a Universal Serial Bus (USB) port, serial port, or other interface methodologies.

Referring to FIGS. 1A and 1B, the interface can be connected to the goniometers and/or dynamometers by one or more wires 165. If so, the wires 165 may be concealed, such as, for example, under a layer of fabric.

In some embodiments, the interface 160, 260 comprises a computer readable medium configured to store the data for later transmission. Common forms of computer-readable media include, for example, magnetic media, optical media, physical media, memory chips or cartridges, or any other medium from which a computer can read. Example computer-readable media may include, but is not limited to, volatile media, non-volatile media, and transmission media. Example tangible, computer-readable recording media include, but are not limited to, solid-state device, RAM, ROM, electrically erasable program read-only memory (EEPROM), flash memory, or other memory technology.

In some cases, the interface 160, 260 contains a processor and memory configured with instructions to monitor and calculate compliance by the subject to a daily rehabilitation regimen. In some cases, the interface 160, 260 is configured to transmit the data to a computing device 700, such as a personal computer (PC) or a mobile computing device, that contains a processor and memory configured with instructions to monitor and calculate compliance by the subject to a daily rehabilitation regimen. Non-limiting examples of suitable mobile computing devices include smartphones and tablets.

The memory in the interface 160, 260 or computing device 700 can have computer-executable instructions stored thereon that, when executed by the processor, cause the processor to monitor and calculate compliance by the subject to a daily rehabilitation regimen. In some cases, the memory in the interface 160, 260 or computing device 700 can have computer-executable instructions stored thereon that, when executed by the processor, cause the processor to generate a cue in the sensory feedback portion 170 when the subject complies with a set range of motion, a set strength, or a combination thereof.

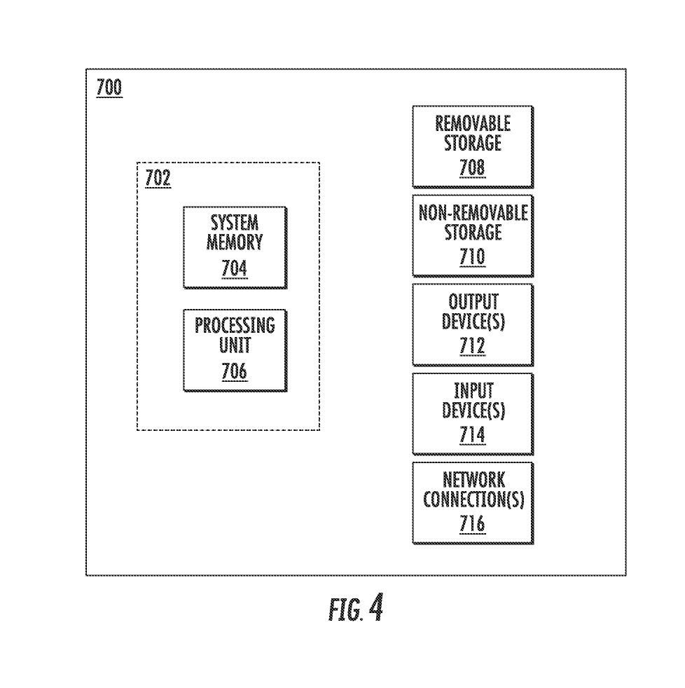

For example, referring to FIG. 4, an example computing device 700 upon which embodiments of the invention may be implemented is illustrated. The computing device 700 may include a bus or other communication mechanism for communicating information among various components of the computing device 700. In its most basic configuration, computing device 700 typically includes at least one processing unit 706 and system memory 704. Depending on the exact configuration and type of computing device, system memory 704 may be volatile (such as random access memory (RAM)), non-volatile (such as read-only memory (ROM), flash memory, etc.), or some combination of the two. This most basic configuration is illustrated in FIG. 7 by dashed line 702. The processing unit 706 may be a standard programmable processor that performs arithmetic and logic operations necessary for operation of the computing device 700.

Computing device 700 may have additional features/functionality. For example, computing device 700 may include additional storage such as removable storage 708 and non-removable storage 710 including, but not limited to, magnetic or optical disks or tapes. Computing device 700 may also contain network connection(s) 716 that allow the device to communicate with other devices. Computing device 700 may also have input device(s) 714 such as a keyboard, mouse, touch screen, etc. Output device(s) 712 such as a display, speakers, printer, etc. may also be included. The additional devices may be connected to the bus in order to facilitate communication of data among the components of the computing device 700. All these devices are well known in the art and need not be discussed at length here.

The processing unit 706 may be configured to execute program code encoded in tangible, computer-readable media. Computer-readable media refers to any media that is capable of providing data that causes the computing device 700 (i.e., a machine) to operate in a particular fashion. Various computer-readable media may be utilized to provide instructions to the processing unit 706 for execution. Common forms of computer-readable media include, for example, magnetic media, optical media, physical media, memory chips or cartridges, a carrier wave, or any other medium from which a computer can read. Example computer-readable media may include, but is not limited to, volatile media, non-volatile media and transmission media. Volatile and non-volatile media may be implemented in any method or technology for storage of information such as computer readable instructions, data structures, program modules or other data and common forms are discussed in detail below. Transmission media may include coaxial cables, copper wires and/or fiber optic cables, as well as acoustic or light waves, such as those generated during radio-wave and infra-red data communication. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 706 may execute program code stored in the system memory 704. For example, the bus may carry data to the system memory 704, from which the processing unit 706 receives and executes instructions. The data received by the system memory 704 may optionally be stored on the removable storage 708 or the non-removable storage 710 before or after execution by the processing unit 706.

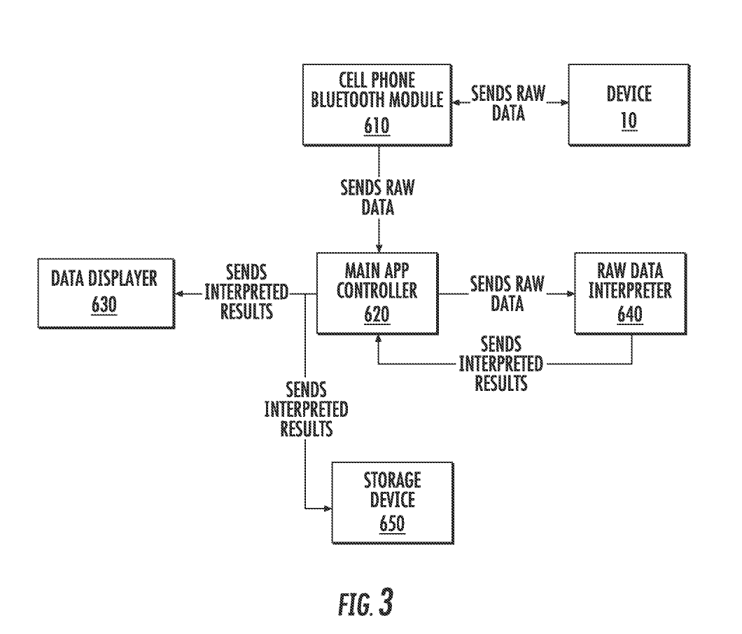

For example, referring now to FIG. 3, a block diagram is shown illustrating an exemplary process whereby the raw data from the rehabilitation monitoring device 10 is transmitted to a wireless module 610 (e.g., Bluetooth®) of the mobile computing device 700, where it then passes to the main application controller 620. From there the raw data is interpreted by a raw data interpreter 640, which sends the interpreted results back to the main application controller 620. The interpreted results are then sent to either a data displayer 630 for the user, a storage device 650 for later transmission or viewing, or both.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

Also disclosed is a method for monitoring rehabilitation by a subject that involves collecting data from a rehabilitation monitoring device worn by the subject during at-home rehabilitation exercise, wherein the rehabilitation monitoring device comprises a goniometer positioned to measure flexion/extension kinematics of the joint and a dynamometer configured to detect flexor strength, extensor strength, or a combination thereof, of the joint; and analyzing the data to determine rehabilitation compliance, progress, or a combination thereof, by the subject. For example, the rehabilitation can involve stroke rehabilitation and/or joint rehabilitation.

A number of embodiments of the invention have been described. Nevertheless, it will be understood that various modifications may be made without departing from the spirit and scope of the invention. Accordingly, other embodiments are within the scope of the following claims.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A device for monitoring rehabilitation exercises by a subject involving a knee joint, comprising:
a) a goniometer configured to measure flexion/extension kinematics of the joint;
b) a wearable attachment portion configured to operably position the goniometer relative to the joint; and
c) an interface configured to receive data from the goniometer.

2-3. (canceled)

4. The device of claim 1, wherein the attachment portion comprises a brace.

5. The device of claim 1, further comprising a sensory feedback portion affixed to the wearable attachment.

6. The device of claim 5, wherein the sensory feedback portion is configured to generate a visible cue, audible cue, haptic cue, or combination thereof, to the subject.

7. The device of claim 1, wherein the goniometer comprises a Hall effect, inductive, magnetoresistive, or potentiometric rotary position sensor.

8. The device of claim 1, wherein the goniometer comprises a force sensitive resistor.

9. The device of claim 1, wherein the goniometer comprises a flexion sensor.

10. The device of claim 9, wherein the flexion sensor comprises a fibre-optic bend sensor.

11. The device of claim 27, wherein the dynamometer comprises a pressure sensor, a force sensitive resistor, or a combination thereof.

12. The device of claim 1, wherein the interface is configured to wirelessly transmit the data to a computing device.

13. The device of claim 1, wherein the computing device is a mobile computing device.

14. (canceled)

15. The device of claim 1, wherein the interface comprises a computer readable medium configured to store the data for later transmission.

16. The device of claim 1, wherein the interface or computing device comprises a processor and memory configured with instructions to calculate compliance by the subject to a daily rehabilitation regimen.

17. The device of claim 16, wherein the processor is configured with instructions to generate a cue in the sensory feedback portion when the subject complies with a set range of motion, a set number of repetitions, a set period of time, or a combination thereof.

18-19. (canceled)

20. The device of claim 27, further comprising a resistance portion that inhibits flexion, extension, or a combination thereof.

21. The device of claim 20, wherein the dynamometer comprises the resistance portion.

22. (canceled)

23. The device of claim 21, wherein the resistance portion is isokinetic.

24. A method for monitoring rehabilitation by a subject, comprising
a) collecting data from a rehabilitation monitoring device worn by the subject during at-home joint rehabilitation exercise, wherein the rehabilitation monitoring device comprises a goniometer positioned to measure flexion/extension kinematics of the joint; and
b) analyzing the data to determine rehabilitation compliance, progress, or a combination thereof, by the subject.

25-26. (canceled)

27. The device of claim 1, further comprising a dynamometer configured to detect flexor strength, extensor strength, or a combination thereof, of the joint, wherein the wearable attachment portion is configured to operably position the dynamometer relative to the joint, and wherein the interface is configured to receive data from the dynamometer.

28. The method of claim 24, wherein the rehabilitation monitoring device further comprises a dynamometer configured to detect flexor strength, extensor strength, or a combination thereof, of the joint.
